(19) United States
(12) Patent Application Publication
(10) Pub. No.: US 2016/0367226 A1
(43) Pub. Date: Dec. 22, 2016

(54) URINE COLLECTION APPARATUS AND RELATED METHODS

(71) Applicants: CAMILLE ROSE NEWTON, BONSALL, CA (US); RAYMOND JOHN NEWTON, BONSALL, CA (US)

(72) Inventors: CAMILLE ROSE NEWTON, BONSALL, CA (US); RAYMOND JOHN NEWTON, BONSALL, CA (US)

(21) Appl. No.: 14/625,469

(22) Filed: Feb. 18, 2015

Related U.S. Application Data
(60) Provisional application No. 61/955,537, filed on Mar. 19, 2014.

(51) Int. Cl. A61B 10/00; A01K 23/00; A61F 13/53; A61B 5/20

(52) U.S. Cl. CPC A61B 10/007; A61B 5/207; A01K 23/005; A61F 13/53; A61B 2560/06; A61B 2503/40; A61F 2013/15146

(57) ABSTRACT

A urine collection device and a moisture-wicking article for use in a system for transporting urine voided from the urethra. The collection device includes an elongated container. The moisture-wicking article is wrapped and secured over an array of side openings in the container, and is disposed in contact with a region surrounding the urethral opening. Urine is drawn from the moisture wicking article through the side openings and into a chamber within the container, in which a plurality of subdivisions of the chamber extend from one end of the container adjacent the array of side openings; a tunnel extends from an output port at the one end of the container; and a collection space is adjacent a closed other end of the container. The chamber subdivisions extend to the collection space so that urine occupying the collection space can be drawn to the output port from the collection space through the tunnel. The moisture-wicking article includes moisture wicking material is embodied in an elongated sheath-like structure that can be wrapped and secured over the array of side openings. An impermeable layer is secured over a portion of the wicking material that is not disposed for contact with the region of a female body surrounding the urethral opening. The impermeable layer extends beyond and apart from a portion of the wicking material that is disposed for making such contact, to thereby form wing-like extensions by which the moisture-wicking article can be gripped. Data is read from a urine test strip inside transparent tubing being used to transport urine from the urine collection device. The quantity of the urine transported from urine collection device is indicated by monitoring the weight of a reservoir into which the urine is transported. The weight is monitored by using a scale disposed beneath the reservoir.

### BACKGROUND OF THE INVENTION

The present invention pertains to collecting and transporting urine away from the body of a person or an animal during various circumstances and in some aspects to sampling collected urine for analysis.

U.S. Pat. No. 8,287,508 discloses urine collection apparatus for use in a system for transporting urine voided from a person or animal by drawing the urine into a moisture wicking article that is disposed in contact with a region of the person or animal surrounding the urethral opening and further drawing the urine into the collection device from the wicking article.

The urine collection device disclosed in said patent includes an elongated container defining a chamber for collecting urine. The container is closed, except for having an array of openings through which urine can be drawn into the chamber and at least one outlet port through which urine can be drawn away from the chamber. An elongated exterior of the container is configured and dimensioned for enabling the moisture-wicking article to be wrapped and secured over the array of openings for enabling the secured moisture-wicking article to be disposed in contact with the region of a female body surrounding the urethral opening.

One embodiment of the system disclosed in said patent includes a urine collection device, a moisture-wicking article, a vacuum pump, a first flexible tube, a second flexible tube and a urine reservoir. The first flexible tube is coupled between the outlet port of the urine collection device and the urine reservoir. The second flexible tube is coupled between the urine reservoir and the vacuum pump. The vacuum pump is adapted for applying a partial vacuum to the outlet port of the urine collection device via the second flexible tube, the urine reservoir and the first flexible tube to thereby cause urine collected in the moisture-wicking article to be drawn through the array of openings in the urine collection device and into the chamber of the urine collection device, and thence to be drawn away from the chamber through the outlet port and the first flexible tube and into the urine reservoir.

### SUMMARY OF THE INVENTION

In various aspects, the invention provides improvements to the urine collection apparatus disclosed in the above-referenced U.S. Pat. No. 8,287,508.

In one aspect, the invention provides a urine collection device having the features recited in the claims.

In another aspect, the invention provides a moisture wicking article having the features recited in the claims.

In further aspects, the invention provides methods of sampling urine for analysis having the features recited in the claims.

Additional features of the present invention are described with reference to the detailed description.

### BRIEF DESCRIPTION OF THE DRAWING

FIG. 1 is a perspective view of one embodiment of a urine collection device according to the invention.

FIG. 2 is a perspective view of a moisture-wicking article according to the invention wrapped and secured over the array of side openings in the urine collection device shown in FIG. 1.

FIG. 3 is a perspective view of one end of the urine collection device shown in FIG. 1 with an end cap removed.

FIG. 3A is a perspective interior view of a cap for the one end of the urine collection device shown in FIG. 1.

FIG. 4 is a perspective view of the other end of the urine collection device shown in FIG. 1 with an end cap removed.

FIG. 4A is a perspective interior view of a cap for the other end of the urine collection device shown in FIG. 1.

FIG. 5 is a schematic view of a urine test strip inserted within transparent tubing being used for transporting urine from a urine collection device.

These figures are not drawn to scale.

### DETAILED DESCRIPTION

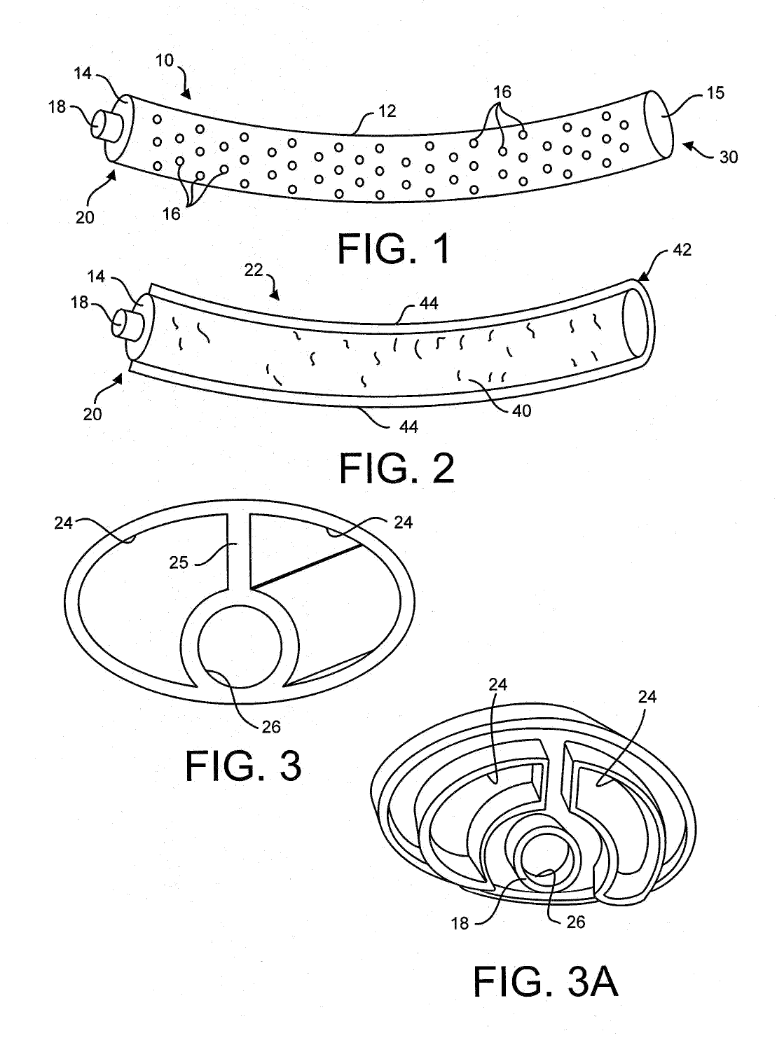

Referring to FIGS. 1 through 4A, one embodiment of a urine collection device 10 according to the invention includes an elongated container 12. The container 12 defines an interior chamber for collecting urine. The container 12 is closed by a first end cap 14 and a second end cap 15, except for having an array of side openings 16 through which urine can be drawn into the chamber and an outlet port 18 at one end 20 of the container 12 through which urine can be drawn away from the chamber.

An elongated exterior of the container 12 is configured and dimensioned for enabling a moisture-wicking article 22 to be wrapped and secured over the array of side openings 16 for enabling the wrapped and secured moisture-wicking article 22 to be disposed in contact with the region of a female body surrounding the urethral opening.

In an alternative embodiment (not shown) a moisture-wicking fabric is sprayed and secured over the array of openings. Spray-on fabric technology, which was developed by Fabrican, Ltd. (www.fabricanltd.com), involves the creation of a liquid suspension which is then sprayed by use of either a spray gun or an aerosol can. The fabric is formed by the cross-linking of fibers which adhere to create an instant non-woven fabric that can be easily sprayed on to any surface.

The interior of the container 12 is configured to define two chamber subdivisions 24, which are divided by a spine 25, a tunnel 26 and a collection space 28. The chamber subdivisions 24 are disposed adjacent the array of side openings 16 and extend from a closed portion of the one end 20 of the container 12. The tunnel 26 extends from the output port 18 and is disposed apart from and approximately parallel with the chamber subdivisions 24. The collection space 28 is adjacent the closed other end 30 of the container 12 for collecting urine drawn into the chamber through the array of side openings 16.

The spine 25 adds strength to the container 12. In one alternative embodiment (not shown) additional chambers are provided by providing additional spines. In another alternative embodiment, the spine is eliminated, whereby only one chamber is provided.

The chamber subdivisions 24 and the tunnel 26 extend to the collection space 28, so that urine occupying the collection space 12 can be drawn to the output port 18 from the collection space 28 through the tunnel 26. In one example, urine is drawn from the collection space 28 through tubing (not shown) that is inserted into the collection space 28 via the output port 18 and the tunnel 26. In another example, urine is drawn from the collection space 28 into exterior tubing connected to the output port 18 without inserting any tubing through the tunnel 26 and the output port 18. In these two exemplary embodiments, the tunnel 26 does not necessarily extend to the collection space 28, although, it is preferable that it does so when the container 18 is formed by extrusion. If the tunnel 26 does not extend to the collection space 28, the container 12 will fill with urine up to the point where the urine reaches the tunnel 26 or tubing inserted through the tubing. It is preferable to evacuate the urine before it accumulates to this point.

When in use, the urine collection device 10 is disposed so that the outlet port 18 is higher than the collection space 28 so that urine is quickly drawn by vacuum pressure away from the collection space 28, rather than accumulating in a pool within the collection space 28.

Referring to FIGS. 3 and 3A, the output port 18 is disposed in the first end cap 14, which is at the one end 20 of the container 12, so that the output port 18 is adjacent the tunnel 26 when the end cap closes the one end of the container 12. Preferably, the output port 18 is shaped and dimensioned as a male fitting for exterior tubing. Alternatively, the output port 18 is shaped and dimensioned as a male fitting for the tunnel 26.

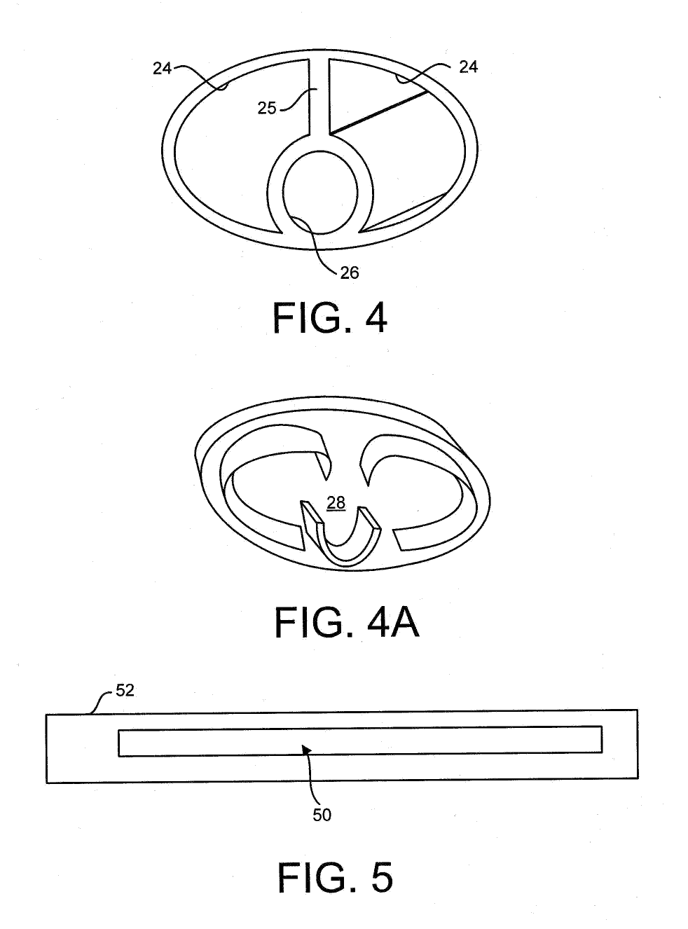

Referring to FIG. 4A, the collection space 28 is in the second end cap 15, which is at the other end 30 of the container 12.

Preferably, both the first end cap 14 and the second end cap 15 are rigid; and are injection molded with a plastic material, such as ABS or Nylon. In various alternative embodiments, the first end cap 14 and/or second end cap 15 is not rigid, is made of some other material, and/or is not injection molded.

In one embodiment, the portion of the container 12 disposed between the two end caps 14, 15 is made of an extruded soft material, such as polyurethane, polyethylene or synthetic rubber. The material is coiled during extrusion to cause the container to have a natural curved shape. The container 12 has a curved shape to enhance a close fit of a wrapped and secured moisture-wicking article 22 to the region surrounding the urethral region of a female body.

In alternative embodiments (not shown) the container 12 is injection molded, has some other shape and/or is not rigid and/or is made of some other material, such as aluminum or a composite of plastic and aluminum and/or some other metal.

Referring to FIG. 2, the moisture-wicking article 22 includes moisture wicking material 40 and an impermeable layer 42, which are embodied in an elongated sheath-like structure. The sheath-like structure is shaped and dimensioned for being wrapped and secured over the side openings 16 of the elongated container 12. A portion of the moisture wicking material 40 is disposed in such structure for contact with the region of a female body surrounding the urethral opening. The impermeable layer 42 is secured over a portion of the wicking material 40 that is not disposed for contact with the region of a female body surrounding the urethral opening.

The impermeable layer 42 extends beyond and apart from the portion of the moisture-wicking material 40 that is disposed for making such contact, to form wing-like extensions 44 of the impermeable layer 42 by which the moisture-wicking article 22 can be gripped without necessarily touching the moisture wicking material 40.

The edges of the moisture-wicking material 40 are heat/pressure sealed to form the sheath, which in one embodiment has one open end, similar to a sock so that the moisture-wicking article fits over the urine collection device like a sock

In an alternative embodiment (not shown), there is a hoop-type handle at one end of the moisture-wicking article to act as a handle to aide when positioning and removing the moisture-wicking article in relation to the urine collection device.

Also, referring to FIG. 1, the exterior of the container 12 is configured for enabling the moisture-wicking article 22 to be wrapped and secured over the array of openings 16 and for enabling the secured moisture-wicking article 22 to be disposed in contact with the region of a female body surrounding the urethral opening. In alternative embodiments, the openings may have many different arrays, shapes and spacings alternative to those of the openings 16 shown in FIG. 1.

Preferably, the array of openings 16 extends throughout an area that is somewhat larger than the area of the moisture-wicking material 40 that is immediately wetted by urine flow from the urethral opening. The area throughout which the array of openings 16 extends should not be too much larger than the immediately wetted area; otherwise excessive air flowing into the chamber through the side openings 16 reduces the partial vacuum within the chamber and thereby reduces the urine collection rate and the efficiency of the urine collection device 10.

Different embodiments of the urine collection device according to the invention are configured for both adult and pediatric applications and for veterinary applications for animals of different species and sizes.

The invention also provides various methods of sampling collected urine for analysis as the urine is being transported from a urine collection device.

In one such method, a urine test strip is inserted into transparent tubing which is used for transporting urine from a urine collection device; and subsequently, data is read through the transparent tubing from the urine test strip. In one embodiment, a camera in a portable communication device, such as a smartphone, an i-phone or some other mobile communication device is used to read the data from the urine test strip by providing an image of the test strip. A smartphone app is available for reading data from urine test strips.

In another such method, a urine test strip is disposed in relation to a transparent urine reservoir into which urine is transported from a urine collection device so that as the urine enters the reservoir the urine flows over the test strip; and subsequently, data is read through the transparent reservoir from the urine test strip. In one embodiment, a camera in a portable communication device, such as a smartphone, an i-phone or some other mobile communication device is used to read the data from the test strip by providing an image of the test strip. A smartphone app is available for reading data from urine test strips.

Referring to FIG. 5, a urine test strip 50 is inserted within a transparent tubing 52 for sampling urine that is used for transporting urine collected from the urine collection device 10. Urine test strips are a common medical product. They are used to test many measures of health. Typically, urine test strips are used by dipping the test strip into a urine sample and observing color/patterns on the strip. The colors correspond to known measurement scales.

The urine test strip 50 can be affixed to the inside of transparent tubing 52 that is used for urine transport. Various adhesives may be used to hold the test strip in place. Preferably, the test strip is inserted within a short segment of the tubing, which typically is less than six inches. The short tube segment has a connector at each end to allow installation and removal from the urine transport tubing. When the test is concluded, the short tubing segment containing the urine test strip can be removed and disposed of.

As urine flows through the tube, it activates the strip. The data read from the urine test strip can be sent to a clinician for analysis. The results of the analysis are sent to a cloud-based address for physician access.

In still another method of sampling collected urine for analysis, urine is transported into a reservoir from a urine collection device; and a scale is disposed beneath the reservoir, the weight of the urine in the reservoir is monitored, and the monitored weight of the urine in the reservoir is processed to provide data indicating the weight of the urine transported into the reservoir from the urine collection device.

The benefits specifically stated herein do not necessarily apply to every conceivable embodiment of the present invention. Further, such stated benefits of the present invention are only examples and should not be construed as the only benefits of the present invention.

While the above description contains many specificities, these specificities are not to be construed as limitations on the scope of the present invention, but rather as examples of the preferred embodiments described herein. Other variations are possible and the scope of the present invention should be determined not by the embodiments described herein but rather by the claims and their legal equivalents.

1. A urine collection device for use in a system for transporting urine voided from a person or an animal by drawing the urine into a moisture-wicking article that is disposed in contact with a region of the person or animal surrounding an urethral opening, and further drawing the urine into the collection device from the moisture-wicking article, the urine collection device comprising:
an elongated container defining a chamber for collecting urine, wherein the container is closed, except for having an array of side openings through which urine can be drawn into the chamber and at least one outlet port at one end of the container through which urine can be drawn away from the chamber;
wherein an elongated exterior of the container is configured and dimensioned for enabling a moisture-wicking article to be wrapped and secured over the array of side openings for enabling a said wrapped and secured moisture-wicking article to be disposed in contact with the region of a female body surrounding the urethral opening;
wherein the interior of the container is configured to define:
a chamber disposed adjacent the array of openings, and extending from a closed portion of the one end of the container,
at least one tunnel extending from the at least one output port and disposed apart from and approximately parallel with the chamber; and
a collection space adjacent the closed other end of the container for collecting urine drawn into the chamber through the array of side openings;
wherein the chamber extends to the collection space of the container, so that urine occupying the collection space can be drawn to the output port from the collection space through the tunnel.

2. A urine collection device according to claim 1, wherein the interior of the container is configured to define the chamber as a plurality of subdivisions of the chamber disposed adjacent the array of openings and extending from a closed portion of the one end of the container.

3. A urine collection device according to claim 1, further comprising:
a moisture-wicking fabric is sprayed and secured over the array of openings.

4. A moisture-wicking article adapted for use with an elongated urine collection device for use in a system for transporting urine voided from a body of a person or an animal by drawing the urine into the moisture-wicking article when said article is disposed in contact with a region of the body surrounding the urethral opening, and drawing the urine into the collection device from the moisture-wicking article, wherein the urine collection device includes an elongated chamber for collecting urine, wherein the container is closed, except for having an array of side openings through which urine can be drawn into the chamber and at least one outlet port through which urine can be drawn away from the chamber, and wherein the exterior of the container is configured and dimensioned for enabling moisture-wicking article to be wrapped and secured over said array of side openings for being disposed in contact with the region of a female body surrounding the urethral opening,
the moisture-wicking article comprising:
moisture wicking material disposed for contact with the region of a female body surrounding the urethral opening; and
an impermeable layer secured over a portion of the wicking material that is not disposed for contact with the region of a female body surrounding the urethral opening.

5. A moisture-wicking article according to claim 4, wherein the impermeable layer extends beyond and apart from the portion of the wicking material that is disposed for making said contact, to form wing-like extensions of the impermeable layer by which the moisture-wicking article can be gripped without necessarily touching the moisture wicking material.

6. A moisture-wicking article adapted for use with an elongated urine collection device for use in a system for transporting urine voided from a body of a person or an animal by drawing the urine into the moisture-wicking article when said article is disposed in contact with a region of the body surrounding the urethral opening, and drawing the urine into the collection device from the moisture-wicking article, wherein the urine collection device includes an elongated chamber for collecting urine, wherein the container is closed, except for having an array of side openings through which urine can be drawn into the chamber and at least one outlet port through which urine can be drawn away from the chamber, and wherein the exterior of the container is configured and dimensioned for enabling moisture-wicking article to be wrapped and secured over said array of side openings for being disposed in contact with the region of a female body surrounding the urethral opening,
the moisture-wicking article comprising:
moisture wicking material embodied in an elongated sheath-like structure for being wrapped and secured over said side openings of a said elongated container; and
an impermeable layer secured over a portion of the wicking material that is not disposed for contact with the region of a female body surrounding the urethral opening.

7. A moisture-wicking article according to claim 6, wherein the impermeable layer extends beyond and apart from the portion of the wicking material that is disposed for making said contact, to form wing-like extensions of the impermeable layer by which the moisture-wicking article can be gripped without necessarily touching the moisture wicking material.

8. A method of sampling urine for analysis, comprising the steps of:
(a) inserting a urine test strip into transparent tubing which is used for transporting urine from a urine collection device; and
(b) subsequently, reading data through the transparent tubing from the urine test strip.

9. A method according to claim 8, wherein step (b) comprises the step of:
(c) using a camera in a portable communication device to read the data from the test strip.

10. A method of sampling urine for analysis, comprising the steps of:
(a) disposing a urine test strip in relation to a transparent urine reservoir into which urine is transported from a urine collection device so that as the urine enters the reservoir the urine flows over the test strip; and
(b) subsequently, reading data through the transparent reservoir from the urine test strip.

11. A method according to claim 10, wherein step (b) comprises the step of:
(c) using a camera in a portable communication device to read the data from the test strip.

12. A method of sampling urine for analysis, comprising the steps of:
(a) transporting urine into a reservoir from a urine collection device;
(b) with a scale disposed beneath the reservoir, monitoring the weight of the urine in the reservoir, and
(c) processing the monitored weight of the urine in the reservoir to provide data indicating the quantity of the urine transported into the reservoir from the urine collection device.
